(19) United States
(12) Patent Application Publication
(10) Pub. No.: US 2009/0254180 A1
(43) Pub. Date: Oct. 8, 2009

(54) CUSTOMIZABLE VERTEBRAL REPLACEMENT ASSEMBLY SYSTEM

(76) Inventors: Walt Pazanowski, Cardiff, CA (US); Timothy Allen Peppers, LaJolla, CA (US)

Correspondence Address: KAUTH , POMEROY , PECK & BAILEY ,LLP, 2875 MICHELLE DRIVE, SUITE 110, IRVINE, CA, 92606, US

(21) Appl. No.: 12/353,910

(22) Filed: Jan. 14, 2009

Related U.S. Application Data
(60) Provisional application No. 61/020,875, filed on Jan. 14, 2008.

(51) Int. Cl. A61F 2/44

(52) U.S. Cl. 623/17.11

(57) ABSTRACT

A customizable vertebral replacement assembly system and methods of using such a system to perform spinal fusions are described. The assembly generally includes a wall that encloses a hollow central core, a plurality of openings arranged in the wall that extend from the exterior or the assembly to the interior of the central core, and at least one stacking armature. The openings disposed in the surface of the wall both allow for the easy trimming and adjustment of the size and shape of the assembly and for bone to grow into the assembly once the spacer is installed in the body, which can help to secure the assembly in place, while the stacking armature allows for the facile interconnection of multiple assemblies.

### CROSS-REFERENCE TO RELATED APPLICATIONS

The current application claims priority to U.S. Provisional Application No. 61/020,875, filed Jan. 14, 2009, the disclosure of which is incorporated herein by reference.

### FIELD OF THE INVENTION

The current invention is directed to a customizable vertebral replacement assembly system for use in vertebral body replacement.

### BACKGROUND OF THE INVENTION

The vertebral column (backbone or spine) is a column of 24 vertebrae, the sacrum, intervertebral discs, and the coccyx situated in the dorsal aspect of the torso, separated by spinal discs. It houses the spinal cord in its spinal canal. Viewed laterally the vertebral column presents several curves, which correspond to the different regions of the column, and are named the cervical, thoracic, lumbar, and pelvic regions.

When the vertebrae of the spine are property articulated with each other, the bodies form a strong pillar for the support of the head and trunk. In addition, the vertebral foramina form a canal that follows the different curves of the column and provides a protective chamber for the spinal cord. However, any deformation of the spine can weaken the architecture of the body and impinge on the spinal cord, impacting nerve function.

Indeed, there are a number of fairly common abnormalities that must be address via surgical intervention, including, for example kyphosis, which is an exaggerated kyphotic (posterior) curvature in the thoracic region, lordosis, which is an exaggerated lordotic (anterior) curvature of the lumbar region, “swayback”, and scoliosis, which involves lateral curvature of the spine and is the most common abnormal curvature occurring in 0.5% of the population. In addition, spinal trauma, either via accident or disease can require the removal or fixation of one or more vertebral bodies or intervertebral discs.

In many of these cases, systems for vertebral body replacement are used in spinal fusion procedures to repair such damaged or incorrectly articulating vertebrae. Conventional spinal fusion techniques employ spacer assemblies that comprise mesh spacer tubes and end caps that can both serve to provide proper spacing between the vertebral bodies and encourage fusion of the vertebrae. These spacers are formed of a surgical grade material and come in a wide-variety of sizes and shapes. In addition, they are usually designed to be trimmed and shaped during the procedure to ensure a better fit to the patient's body. However, thus far little thought has been put into making the customization of these assemblies more facile. This is particularly important given the complex nature of spinal surgery and the growing concern in the field over the time under anesthesia required to perform spinal surgeries. In short, every moment saved improves the likely outcome of the surgery.

Accordingly, there exists a need for improvements in the field of vertebral replacement assemblies, and particularly a need for assemblies that may be easily tailored to the specific geometry and dimension of the patient's body.

### SUMMARY OF THE INVENTION

The current invention is directed to a customizable vertebral replacement assembly system that may be modified in situ either by cutting, bending or stacking together the assemblies.

In one embodiment, the vertebral replacement assembly generally comprises a wall enclosing a hollow body, the wall being configured to include a plurality of openings that connect the exterior of the assembly to the interior of the assembly.

In another embodiment, the plurality of openings along the watt of the vertebral replacement assembly are shaped and disposed in a pattern to provide maximum flexibility in contouring the final assembly. In one such embodiment, the openings take the form of either a series of staggered crosses or a cobblestone pattern of squares.

In still another embodiment the vertebral replacement assemblies of the current invention are stackable. In one such embodiment, the assemblies are provided with armatures at one end capable of interlocking with adjacent cages allowing for multiple cages to be stacked. In such an embodiment these armatures can be removed or sheared off should stacking not be required. In another such embodiment, the assemblies may be formed with variable cross-sections such that they can be overlapped and joined together via set screws.

In yet another embodiment the vertebral replacement assembly has walls of about 1.5 mm.

In still yet another embodiment, the vertebral replacement assembly may have any suitable cross-section, such as, for example, circular, ovoid or square.

In still yet another embodiment, the vertebral replacement is made by metal injection molding.

In still yet another embodiment, the vertebral replacement assembly system further includes a specialized shaping tool to allow for the creation of a desired curvature in the assembly. In such an embodiment the shaping tool includes a contoured molding device and an internal insert, such as a mandrel/snake, that would be inserted into the cage to prevent the cross-section of the cage from being deformed during the bending process.

The invention is also directed to methods of stabilizing the spine and correcting spinal curvature using the vertebral replacement assembly system of the invention.

### BRIEF DESCRIPTION OF THE DRAWINGS

The description will be more fully understood with reference to the following figures, which are presented as exemplary embodiments of the invention and should not be construed as a complete recitation of the scope of the invention, wherein:

FIGS. 1a and 1b provide schematic diagrams of an exemplary embodiment of the vertebral replacement assemblies of the current invention;

FIGS. 2a and 2b provide schematic diagrams of exemplary opening shapes and patterns for the vertebral replacement assemblies of the current invention;

FIG. 3 provides a schematic of an exemplary vertebral replacement assembly shaping toot in accordance with the current invention; and

FIG. 4 provides a schematic showing a stabilizing insert for use with when shaping a vertebral replacement assembly with the vertebral replacement assembly shaping toot shown in FIG. 3.

### DETAILED DESCRIPTION OF THE INVENTION

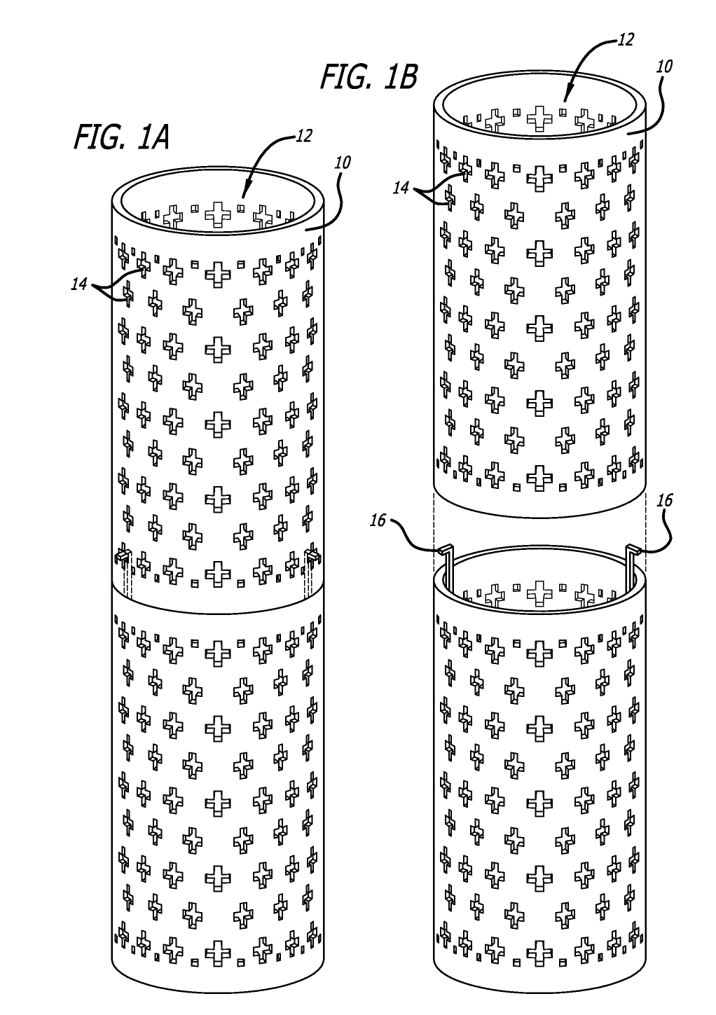

The current invention is directed to a customizable vertebral replacement assembly system. FIGS. 1 to 4 illustrate various embodiments of the vertebral replacement assembly system, which will be referred to herein after as either the assembly or cage. Such reference is for ease of description and is not intended to limit the scope of the invention.

FIGS. 1a and 1b illustrate an exemplary embodiment of the assembly in accordance with the current invention. As shown, the assembly generally includes a wall (10) that encloses a hollow central core (12), a plurality of openings (14) arranged in the wall that extend from the exterior or the assembly to the interior of the central core, and at least one stacking armature (16). The openings disposed in the surface of the wall both allow for easy trimming and adjustment of the size and shape of the assembly, and for bone to grow into the assembly once the spacer is installed in the body, which can help to secure the assembly in place. The stacking armature allows for the facile interconnection of multiple assemblies.

The openings (14) and the pattern of openings (18) provided in the wall of the assembly may take any number of different shapes and configurations suitable for the specific use of the assembly. However, in a preferred embodiment the openings and the pattern of openings are designed to allow a surgeon greatest flexibility in customizing the contour and height of the assembly to suit the surgical site. Specifically, the openings and patterns of openings are preferably designed to allow a surgeon to cut between the openings in multiple directions with a minimum of effort and thereby provide a high level of customizability to the overall shape and height of the assemblies.

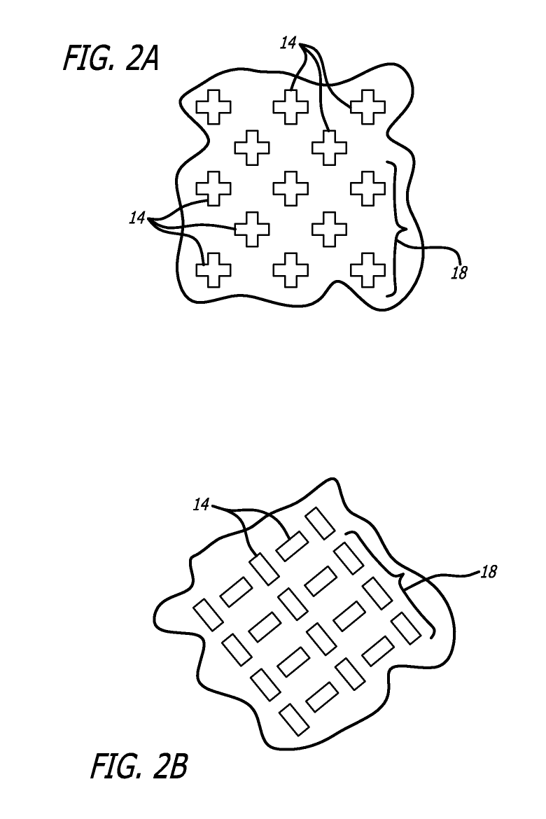

Two particularly preferred embodiments of the opening geometries are provided in FIGS. 2a and 2b. In the embodiment of FIG. 2a, the opening has a cross shape. As shown, in this embodiment the cross shapes are staggered, that is each row of crosses is overlapped by the crosses in the rows above and below. This geometry allows for very dense packing of the openings, and the arms on the crosses also allows the surgeon to cut away and into each opening from multiple angles. An alternative arrangement of openings is provided in FIG. 2b. In this embodiment, the openings have a simple rectangular shape, but they are disposed on the surface in a cobblestone-paving pattern, allowing for a greater number and variety of possible geometries to be produced by cutting between the openings.

Although specific opening sizes and relative positions are shown in the figures, it should be understood that these embodiments are merely provided for illustration. The openings may be sized and spaced as desired, however, in a typical embodiment the dimension of the feature from its center to its edge is on the order of a few millimeters (˜2.5 mm). The spacing dimension between features is similarly on the order of a few millimeters (˜1 to 3 mm.) The only constraint on the size and spacing of the features is that the wall of the assembly retain sufficient structural integrity to provide the requisite support to the spine.

The relative dimensions of the assembly are also variable to conform to the needs of the specific operation. However, in a typical embodiment the assembly will have an outer diameter of about 12 mm and a wall thickness of about 1.5 mm. Although thicker or thinner walled assemblies may be produced, a wall thickness of less than about 1.5 mm will provide sufficient strength to the assembly while simultaneously allowing the wall of the assembly to be cut an shaped using a conventional pair of side-cut pliers. The length of the cage is likewise customizable depending on the need, and can vary from as small as a few millimeters to a few centimeters. Typical length dimensions for such assemblies are from, for example, 4 mm to 25 mm. Finally, although the cross-section of the assemblies illustrated herein are circular, it should be understood that any cross-section suitable for use in stabilizing the spine may be used, including for example, ovoid, square, octagonal, hexagonal, etc.

As discussed briefly above, the assemblies of the current invention also include at least one stacking armature or lug (16) disposed on at least one end of the assembly for interconnecting two adjacent assemblies together. The presence of such an armature allows for the limitless expansion of the assemblies of the current invention such that larger overall assemblies can be created without requiring the insertion of a single large construct into the body. Moreover, such expandability allows for the use of shorter base assemblies that could then be expanded into larger overall assembly structures such that fewer instances would arise requiring the base assembly to be cut down, which is both time consuming and sometimes requires repeated insertion and removal of the assembly during the procedure.

Turning to the structure of these stacking armatures, as shown in FIG. 1a in their simplest form the armatures consist of a simple “L” shaped hook that is integrally connected to the wall of the assembly. The armatures are designed to cooperatively engage a feature on either the exterior or interior surface of an adjacent assembly to securely interconnect the two assemblies. In a preferred embodiment, as shown in FIGS. 1a and 1b, a plurality of armatures are integrally connected to the interior surface of the wall of at least one end of each of the assemblies, and are each dimensioned to cooperatively engage one of the openings of adjacent assembly to securely interconnect the two assemblies. Although the thickness of the armatures may vary depending on the requirements of the particular surgery, in a preferred embodiment the armatures are dimensioned to allow easy removal by a standard pair of pliers or cutters should no expansion of the assembly be required.

During use the two assemblies are brought together such that the armatures align with the interconnecting features on the adjacent assembly. The simplicity of the interconnection, and in particular the fact that no screws or other fixation means are required to interconnect assemblies, allows for the facile modification of the final assembly shape without many of the risks inherent with conventional vertebral replacement assemblies. In addition, should the armatures not be required, they may be removed either prior to or after insertion of the assembly using a conventional pair of surgical pliers or cutters without disturbing the placement of the assembly.

Although in the figures armatures are located on only one side of the each of the assemblies, it should be understood that armatures can be integrated into both sides of each of the assemblies such that both assemblies may cooperatively engaged each other, thereby increasing the number of interconnections between the assemblies. Likewise, although a simple “L” shaped clip is shown in the figures, it should be understood that any other armature design capable of cooperatively engaging a feature of an adjacent cage, and preferably an opening of an adjacent cage, may be incorporated into the assemblies of the current invention, such as, for example, a spring connection, a frictional connection, and a ratchet connection, etc. In addition, although the preferred clip embodiments shown in the figures attach through a simple hook interconnection, additional securing means may be incorporated into armatures, including, for example, set-screws, bolts, pins, frictional fittings, ratchets, etc. to provide additional security to the interconnection of the assemblies.

Finally, although the interconnected assemblies shown in the figures have the same cross-sectional dimension, assemblies having different cross-sectional dimensions may be selected such that the interconnected assemblies at least partially nest within each other. In such an embodiment, the securing connection provided by the armatures may be enhanced by also providing holes through which set-screws or other fixation devices may directly interconnect the two assemblies.

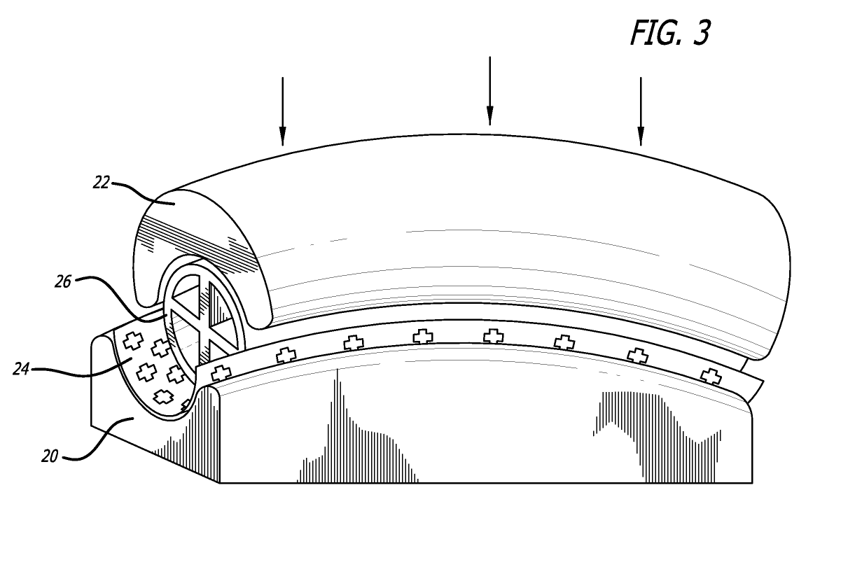

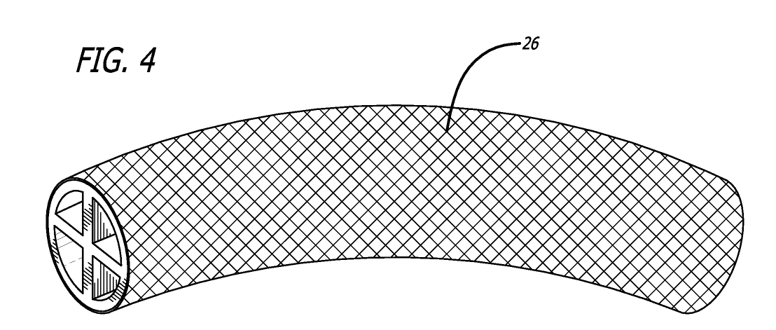

In addition to the vertebral replacement assembly itself, the current invention is also directed to an assembly system, including a shaping tool designed to provide a controlled means of contouring the assemblies to the patient's spine. The first element of the shaping tool of the current invention, as shown in FIG. 3, would be a hand-held compression tool having cooperatively curved and nesting troughs (20 & 22) dimensioned to accept assemblies (24) having a variety of cross-sections and lengths. The second element of the shaping tool, as shown in FIG. 4, would be an interior support snake or mandrel (26) designed to be inserted into the hollow central core of the assembly to prevent the cross-section of the cage from being deformed during the shaping process. Although in the embodiment of the shaping tool shown in FIGS. 3 and 4 the insert and trough portions of the shaping tool are shown as being independent pieces, it should be understood that these two elements of the shaping tool may be integrated into a single-piece hand-tool.

During operation the insert (26) would be placed into position in the interior of the assembly (24). The assembly would then be placed into the lower trough (20) and the upper trough (22) would be brought to bear against the assembly compressing the assembly therebetween. The troughs would preferably have a curvature that ranges from at least 9 to 13 degrees. Additional curvature could be induced in the assembly by pliers or other tools as needed. In such operations the mandrel/snake insert would remain in the assembly during shaping to provide cross-sectional support to the assembly.

The above discussion has focused on the structures of the vertebral replacement assembly system and the proper operation of those elements. However, the current invention is also directed to a method of fusing a spine using the vertebral replacement assemblies described herein. In one such embodiment the surgical site would first be evaluated. After evaluation, the assembly would be shaped as desired, including bending, stacking and/or trimming the assembly/assemblies as required. Such shaping may also include the removal of unused armatures from the assembly/assemblies. Once the proper assembly contour had been obtained the assembly would be inserted into the space as desired.

Finally, it is contemplated that the components of the vertebral replacement assembly system of the present invention can be formed from any suitable biocompatible material, including metals such as titanium and titanium alloys. Although any manufacturing technique may be used to produce the assemblies of the current invention, in one preferred embodiment, the assemblies are formed using a metal injection molding technique. In such an embodiment, a mixture of powdered metal and binder are held together in a mold and heated up. Using such a process it is possible to create final parts without additional costly processing, such as, for example deburring. In addition, the lack of additional machining reduces the risk that the physical properties of the underlying material will be altered due to mechanical stress. It is further contemplated that system of the present invention can be used in a variety of procedures in a number of different applications. In fact, the present system has applicability for any operation where an artificial spinal spacing element may be desired.

Although specific embodiments of the vertebral replacement assembly system of the current invention are described herein, it is expected that persons skilled in the art can and will design alternative embodiments of the assembly and associated tools that are within the scope of the above description either literally or under the Doctrine of Equivalents.

What is claimed is:

1. A vertebral replacement assembly system for use in a spinal fusion comprising:
an enclosure having first and second ends, and having a wall configured to enclose a hollow interior;
a plurality of openings disposed throughout said wall, said openings being configured to connect an exterior of said enclosure to said hollow interior; and
at least one armature disposed on at least one of said ends and configured to engage and interconnect said assembly with an adjacent assembly.

2. The vertebral replacement assembly system of claim 1, wherein the wall has a thickness of around 1.5 mm.

3. The vertebral replacement assembly system of claim 1, wherein the height of the assembly is from between about 6 to 18 millimeters.

4. The vertebral replacement assembly system of claim 1, wherein the diameter of the assembly is from between about 10 to 15 millimeters.

5. The vertebral replacement assembly system of claim 1, wherein the plurality of openings are configured to have a shape selected from the group consisting of crosses and rectangles.

6. The vertebral replacement assembly system of claim 1, wherein the plurality of openings are disposed in a pattern selected from the group consisting of staggered and cobblestone.

7. The vertebral replacement assembly system of claim 1, wherein the plurality of openings are substantially equally spaced along said wall.

8. The vertebral replacement assembly system of claim 1, wherein the assembly has a cross-section selected from the group consisting of circular, ovoid, square, octagonal and hexagonal.

9. The vertebral replacement assembly system of claim 1, wherein the assembly comprises a plurality of said armatures.

10. The vertebral replacement assembly system of claim 1, wherein the armatures are disposed on both ends of said assembly

11. The vertebral replacement assembly system of claim 1, wherein the at least one armature is disposed to interconnect with at least one opening on the adjacent assembly.

12. The vertebral replacement assembly system of claim 1, wherein the at least one armature engages the adjacent assembly through a fixation mechanism selected from the group consisting of a clip connection, a ratchet connection, a frictional fitting and a spring mechanism.

13. The vertebral replacement assembly system of claim 12, wherein the at least one armature is further interconnected to the adjacent assembly through an additional fixation mechanism.

14. The vertebral replacement assembly system of claim 13, wherein the additional fixation mechanism is selected from the group consisting of a screw, bolt and pin.

15. The vertebral replacement assembly system of claim 1, wherein the armature is attached to the inner wall of the assembly and interconnects through the inner wall of the adjacent assembly.

16. The vertebral replacement assembly system of claim 1, wherein the assembly and the adjacent assembly have different cross-sectional dimensions, such that the walls thereof at least partially overlap while interconnected.

17. The vertebral replacement assembly system of claim 16, wherein the assembly and the adjacent assembly further include at least one cooperative opening in each of the walls thereof such that at least one fixation anchore may be engaged between the assemblies.

18. The vertebral replacement assembly system of claim 17, where the at least one fixation anchor is selected from the group consisting of screw, bolts and pins.

19. The vertebral replacement assembly system of claim 1, further including a shaping tool comprising:
a compression tool having two curved troughs defining matching arcs and arranged such that when brought together they cooperatively form a hollow tube having an inner cross-sectional shape and dimension to allow for the insertion of an assembly therebetween,
an insertion tool having an outer cross-sectional shape and dimension such that said insertion tool may be inserted into the hollow interior of said assembly in supportive relation to the interior surface thereof; and
wherein the curved troughs of the compression tool may be compressed together to impart the arc of thereof to an assembly inserted therebetween.

20. The vertebral replacement assembly system of claim 19, wherein the curved troughs of the compression tool describe an arc having an angle from between about 9 and 13 degrees.

21. The vertebral replacement assembly system of claim 19, wherein the shaping tool is an integrated hand-held device.

22. The vertebral replacement assembly system of claim 19, wherein the compression tool and insertion tool are separate devices.

23. The vertebral replacement assembly system of claim 19, wherein the insertion tool is one of either a snake or mandrel.

24. A method of performing a spinal fusion comprising inserting the vertebral replacement assembly of claim 1 into place between two adjacent vertebral bodies.
